(19) United States
(12) Patent Application Publication
(10) Pub. No.: US 2007/0219263 A1
(43) Pub. Date: Sep. 20, 2007

(54) Metronidazole/azelaic acid compositions for the treatment of rosacea

(75) Inventor: William Pilgrim, Valbonne (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 11/723,378

(22) Filed: Mar. 19, 2007

Related U.S. Application Data
(63) Continuation of application No. PCT/FR05/02281, filed on Sep. 14, 2005.

(30) Foreign Application Priority Data
Sep. 17, 2004 (FR) 0409879

(51) Int. Cl. A61K 31/4164; A61K 31/19

(52) U.S. Cl. 514/396; 514/574

(57) ABSTRACT

Pharmaceutical compositions useful for the treatment of rosacea, in particular for the treatment of the inflammatory component thereof, contain synergistically effective amounts of each of (i) metronidazole or derivative or salt thereof and (ii) azelaic acid or derivative or salt thereof.

### CROSS-REFERENCE TO PRIORITY/PCT APPLICATIONS

This application claims priority under 35. U.S.C. § 119 of FR 04/09879, filed Sep. 17, 2004, and is a continuation of PCT/FR 2005/002281, filed Sep. 14, 2005 and designating the United States (published in the French language on Mar. 30, 2006 as WO 2006/032759 A1; the title and abstract were also published in English), each hereby expressly incorporated by reference in its entirety and each assigned to the assignee hereof.

### BACKGROUND OF THE INVENTION

1. Technical Field of the Invention

The present invention relates to pharmaceutical compositions for the treatment of rosacea, and more particularly of the inflammatory component of this condition. This invention also relates to the use of such compositions in the formulation of medicaments for the treatment of rosacea.

2. Description of Background and/or Related and/or Prior Art

Rosacea is a common, chronic and progressive inflammatory dermatosis related to vascular relaxation. It mainly affects the central part of the face and is characterized by redness of the face or hot flushes, facial erythema, papules, pustules and telangiectasia. In serious cases, particularly in men, the soft tissue of the nose may swell and produce a bulbous swelling known as rhinophyma.

Rosacea generally occurs from the ages of 25 and 70, and it is much more common in individuals with a light complexion. It affects more particularly women, although this condition is generally more serious in men. Rosacea is chronic and persists for years with periods of exacerbation and remission.

Rosacea was originally called “acne rosacea” because its papules (points of slight raising of the skin) and its inflammatory pustules (pus scabs) greatly resemble those of common acne. In contrast with common acne, the aetiology of which is based at the same time on abnormal keratinization, an increase in sebum production and a bacterial information, the inflammation of rosacea is vascular in nature and is poorly understood. The result of this facial vascular anomaly is a permanent oedema of the dermis which may accompany an increased colonization with Demodex folliculorum, a mite usually found in the follicles of the face.

According to various studies, Demodex folliculorum is thought to have an aetiological role in rosacea (Erbagi et al., 1998, Int. J. Dermatol., Vol. 37, pages 421-425;. Purcell et at., 1996, J. Am. Acad. Dermatol., Vol.15, pages 1159-1162;. Sibenge et al., 1992, J. Am. Acad. Dermatol., Vol. 26, pages 590-593). It appears that Demodex folliculorum causes or aggravates inflammatory reactions, reflected by papules and pustules, by blocking the pilosebaceous follicles of the face (Roihu et al., 1998, J. Cutan. Pathol., Vol. 25, pages 550-552). This parasite is, moreover, thought to trigger a humoral immune response (Nunzi et al., 1980, Br. J. Dermatol., Vol. 103, pages 543-551; Manna et al., 1982, Br. J. Dermatol., Vol. 107, pages 203-203).

The pathology of rosacea is poorly understood. Many factors may be involved without necessarily inducing this condition. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pilori.

Rosacea develops in 4 stages, but passage through all the stages is not obligatory:
stage 1 of vascular relaxation (at about 20 years old). The patients have sudden flushes of paroxystic redness of the face and neck, with hot sensations, but no systemic signs. After the attacks, the skin of the face returns to normal. These sudden flushes are triggered by changes in temperature (sometimes leading to thermophobia), and the intake of hot drinks or alcohol;
stage 2 of erythemato-telangiectasia (at about 30 years old). The cheekbone areas are diffusely red. Dilated capillaries constituting standard rosacea are observed therein. Unlike stage 1, the redness is permanent. In addition to the cheeks, the chin and the middle part of the forehead may be affected;
stage 3 of papulo-pustules (at about 40 years old). Papules and pustules a few millimeters in diameter develop on the background of erythema, without associated comedones. This dermatitis may be very extensive, sometimes up to the bald part of the scalp in men, but is absent from the area around the mouth and the eyes. The patients complain of sensitive skin, with subjective intolerance to the majority of topical products and greasy cosmetics;
stage 4 of rhinophyma (at about 50 years old or later). This late phase mainly affects men, unlike the other stages. The nose is increased in volume and diffusely red and the follicular orifices are dilated. The skin gradually thickens.

Admittedly, the minor forms of rosacea may be treated with active agents such as anti-seborrheic agents and anti-infective agents, for example benzoyl peroxide or retinoic acid. As regards the most diffuse forms of the condition, they respond well to general antibiotic therapy with cyclines. However, these treatments have unpleasant side effects for the patient, such as irritation or intolerance phenomena. In fact, due to the multifactorial aspect of rosacea, there are huge numbers of treatments for this condition, but the search continues for an effective treatment that is without risk for the patient.

### SUMMARY OF THE INVENTION

The pharmaceutical compositions according to the invention comprise at least two active ingredients: metronidazole and azelaic acid.

Metronidazole, or 2-(2-methyl-5-nitroimidazolyl)ethanol, is known in the prior art for its anti-bacterial, anti-parasitic and anti-protozoin properties. It exerts a selective toxicity with respect to anaerobic microorganisms and also hypoxic cells. In the latter, metronidazole is reduced to derivatives capable of impairing the DNA structure of these cells. The assignee hereof has recently demonstrated the advantageous properties of metronidazole on skin inflammation, and more particularly on the inflammatory component in rosacea.

Azelaic acid, or 1,7-heptanedicarboxylic acid, is known in the prior art for its anti-bacterial and comedolytic properties.

Widely recognized as a reference anti-acne agent (product Skinoren (TM) based on 20% azelaic acid, marketed by Schering (TM)), it has also been described in the prior art as having pharmacological properties for the treatment of rosacea (Carmichael A. J., Robinson L. et al.—“Topical azelaic acid in the treatment of rosacea,” J. Dermatol. Treatm., 4, suppl. 1:. S19-S22, 1993.).

Azelaic acid has a bacteriolytic activity that is different from metronidazole, due to the fact that it acts in particular as a mitochondrial oxydoreductase inhibitor (G. Ital. Dermatol. Venereol., 1989. October; 124. (10): 455-63, Passi S; Picardo M; De Luca C; Nazzaro-Porro M), and as a 5-alpha-reductase inhibitor (British Journal of Dermatology, 1988. No, 119(5): 627-32., Stamatiadis, D; Bulteau-Portois, MC; Mowszowicz, I.).

Recently, WO 2003/061766. would suggest topical compositions containing active ingredients used alone or in combination with a mixture of ethyllinoleic acid and triethylcitric acid.

Metronidazole and azelaic acid are indicated among a list of active agents that may be included in said pharmaceutical compositions. However, no embodiment discloses a composition comprising metronidazole and azelaic acid, in particular for the treatment of rosacea.

Even more recently, WO 2004/022046 discloses a composition comprising metronidazole and azelaic acid for the treatment of rosacea. In the single example of this application, the concentrations of active ingredients used (20% azelaic acid and 0.75% metronidazole) are identical to the concentrations found in the corresponding pharmaceutical products currently on the market (20% Skinoren (TM) and 0.75% Rozex (TM)). However, it is not indicated that the combination of metronidazole with azelaic acid has a synergistic effect at such concentrations. Moreover, the high azelaic acid content in the composition is liable to be responsible for the following side effects: local irritations, itching or redness, or even gastrointestinal problems such as nausea, vomiting, loss of appetite or abdominal pain.

Considering the above, the present invention provides pharmaceutical compositions comprising metronidazole and azelaic acid which show an improved effectiveness in the treatment of rosacea, and also the formulation of such compositions into medicaments.

Thus, it has now been discovered that, surprisingly, the combination of metronidazole with azelaic acid, in appropriate concentrations, provides a synergistic effect in the treatment of a skin inflammation, and in particular of the inflammatory component in rosacea.

### DETAILED DESCRIPTION OF BEST MODE AND SPECIFIC/PREFERRED EMBODIMENTS OF THE INVENTION

Thus, the present invention features pharmaceutical compositions for the treatment of rosacea, comprising, formulated into a pharmaceutically acceptable medium, at least metronidazole, or one of its pharmaceutically acceptable derivatives, and azelaic acid, or one of its pharmaceutically acceptable derivatives, at concentrations such that a synergistic effect from the metronidazole and the azelaic acid is observed. Preferably, the concentration of metronidazole is equal to 0.75% by weight of the total weight of the composition, and the concentration of azelaic acid is less than 10% by weight of the total weight of the composition.

In addition, this invention features formulating the subject compositions into medicaments for the treatment of rosacea.

Metronidazole and azelaic acid are present, in the compositions according to the invention, in concentrations such that they act in synergy to confer on the compositions a therapeutic effect greater than the theoretical effect obtained by adding the effects obtained by each of the two active agents taken separately.

In the compositions according to the invention, the metronidazole is included at concentrations of from 0.001% to 20% by weight relative to the total weight of the composition, and more preferably from 0.1% to 2%, in particular 0.75%, 1%, 1.5% or 2%.

Throughout the entire present text, unless otherwise specified, it is understood that, when concentration ranges are given, they include the upper and lower limits of said range.

In practice, the concentration of azelaic acid is less than 10% by weight of the total weight of the composition. It is preferably from 0.5% to 8% by weight of this total weight %, and more preferably from 1. % to 7% by weight %, in particular 2%, 3%, 6% or 7%.

It has thus been demonstrated that, surprisingly, low doses of azelaic acid, less than 10% by weight, combined in the same composition with 0.75% by weight of metronidazole, have an effectiveness in the region of that of a combination of azelaic acid at a concentration equal to 20% by weight, with 0.75% by weight of metronidazole.

The compositions of the present invention are dermatological compositions for topical administration to the skin.

The subject compositions provide the following advantages, compared with the prior art, in the case of the treatment of skin conditions:
the compositions which contain a combination of metronidazole and azelaic acid at concentrations such that a synergistic effect from the metronidazole and the azelaic acid is substantially more effective than a composition comprising metronidazole alone or azelaic acid alone,
the administration of a combination of azelaic acid and metronidazole makes it possible to shorten the treatment period,
the compositions which contain a mixture of metronidazole and azelaic acid, for topical application, make it possible to administer a lower dose of azelaic acid or even of metronidazole, and thus to decrease the side effects associated, firstly, with metronidazole and, secondly, with azelaic acid,
the administration (whether regime or regimen) of a combination of metronidazole and azelaic acid decreases in particular the irritation side effects of azelaic acid on sensitive skin, such as skin affected, in addition, by psoriasis, thus increasing the tolerance of the metronidazole treatment.

The present invention also features pharmaceutical compositions as defined above, that exhibit a synergistic effect from metronidazole and azelaic acid in the treatment of skin vascularization disorders engendered by rosacea.

The expression “treatment of skin vascularization disorders” means, according to the present invention, the treatment and/or the prevention of such a disorder.

The expression “treatment of rosacea” means, according to the present invention, the treatment and/or prevention of rosacea, at one or more of the stages described above.

According to a first embodiment of the invention, the administration of the subject compositions is for the treatment of the first stage of rosacea.

According to a second embodiment of the invention, the administration of the subject compositions is for the treatment of the second stage of rosacea.

According to a third embodiment of the invention, the administration of the subject compositions is for the treatment of the third stage of rosacea.

According to a fourth embodiment of the invention, the administration of the subject compositions is for the treatment of the fourth stage of rosacea.

According to a preferred embodiment, the composition contains 0.001% to 20% by weight of metronidazole 0.5% to 8% by weight of azelaic acid, preferably from 0.1% to 2% by weight of metronidazole and 1. % to 2% by weight of azelaic acid, expressed as percentage by weight relative to the total weight of the composition, in particular the composition contains 0.75%, 1%, 1.5% or 2% by weight of metronidazole and 2%, 3%, 6% or 7% by weight of azelaic acid, expressed as percentage by weight relative to the total weight of the composition.

Of course, the present invention relates, in addition to the administration of metronidazole and of azelaic acid, to the administration of derivatives thereof. The term “derivatives” means compounds that differ from metronidazole and from azelaic acid by virtue of the substitution, addition or deletion of one or more chemical groups and which exhibit substantially the same activity, and also their salts with a pharmaceutically acceptable acid or base.

Advantageously, the compositions of the invention comprise, in addition to the metronidazole and the azelaic acid, at least one other therapeutic agent capable of increasing the effectiveness of the treatment. Non-limiting examples of such agents include antibiotics, anti-bacterial agents, anti-viral agents, anti-parasitic agents, anti-fungal agents, anaesthetics, analgesics, anti-allergic agents, retinoids, free-radical scavengers, anti-pruriginous agents, keratolytics, anti-seborrheic agents, anti-histamines, sulfides, immunosuppressant or anti-proliferative products, or mixtures thereof.

The compositions of the invention may also comprise any additive normally employed in the pharmaceutical or dermatological field, and compatible with metronidazole and azelaic acid.

Particularly exemplary are sequestering agents, antioxidants, sunscreens, preservatives, for example DL-alpha-tocopherol, fillers, electrolytes, humectants, dyes, the usual inorganic or organic bases or acids, fragrances, essential oils, cosmetic active agents, moisturizers, vitamins, essential fatty acids, sphingolipids, self-tanning compounds such as DHA, agents that sooth and protect the skin such as allantoin, propenetrating agents, gelling agents, or mixtures thereof. Of course, one skilled in this art will take care to select this or these possible additional compound(s), and/or the amounts thereof, in such a manner that the advantageous properties of the compositions according to the invention are not, or are not substantially, impaired.

The additives may be present in the subject compositions in a proportion of from 0% to 20% by weight relative to the total weight of the composition.

As examples of sequestering agents, representative are ethylenediaminetetraacetic acid (EDTA) and derivatives or salts thereof, dihydroxyethylglycine, citric acid, tartaric acid, or mixtures thereof.

Examples of preservatives are benzalconium chloride, phenoxyethanol, benzyl alcohol, diazolidinylurea, parabens, or mixtures thereof.

As examples of humectants, representative are glycerol and sorbitol.

The compositions of the invention may contain one or more propenetrating agents in preferred concentrations ranging from 0% to 20%, and more preferably ranging from 0.6% to 3% by weight relative to the total weight of the composition. Among the propenetrating agents, exemplary are compounds such as propylene glycol, dipropylene glycol, propylene glycol diperlargonate, lauroglycol and ethoxydiglycol.

Advantageously, the compositions according to the invention may also contain one or more surfactants in preferred concentrations ranging from 0% to 10%, and more preferably ranging from 0.1% to 2% by weight to the total weight.

The compositions of the present invention may be in any of the pharmaceutical forms normally employed for topical application, in particular in the form of aqueous, aqueous-alcoholic or oily solutions, dispersions of the lotion type, aqueous, anhydrous or lipophillc gels, emulsions with a liquid or semi-liquid consistency of the milk type, obtained by dispersion of a fatty phase in an aqueous phase (O/W emulsions) or conversely (W/O emulsions), or suspensions or emulsions with a soft, semi-liquid or solid consistency of the cream, gel or ointment type, or else microemulsions, microcapsules, microparticles or vesicular dispersions of ionic and/or nonionic type.

Preferably, the creams may be formulated from a mixture of mineral oil, or from a mixture of beeswax and of water which emulsifies instantaneously, to which metronidazole, dissolved in a small amount of oil such as almond oil, is added.

Preferably, the creams may be formulated from a mixture of mineral oil, or from a mixture of beeswax and of water which emulsifies instantaneously, to which metronidazole and azelaic acid, dissolved in a small amount of oil such as almond oil, are added.

Preferably, the lotions may be prepared by dissolving the metronidazole and the azelaic acid in a high molecular mass alcohol, such as polyethylene glycol.

The ointments may be formulated by mixing a solution of metronidazole and of azelaic acid in an oil such as almond oil, into heated paraffin, and then allowing the mixture to cool.

The gels may preferably be prepared by dispersing or dissolving the metronidazole and the azelaic acid, in an appropriate ratio, in a gel of carbomer, poloxamer or cellulose-based type.

Examples of compositions according to the invention are those comprising an active phase containing (expressed as percentage by weight):
0% to 10%, preferably 0% to 2%, in particular 0% to 0.5%, of surfactant;
0% to 20%, preferably 0% to 10%, in particular 2% to 5%, of propenetrating agent;
0.001% to 20%, preferably 0.1% to 2%, of metronidazole;
0.5% to 8%, preferably 1% to 7%, of azelaic acid; and an aqueous phase comprising a gelling agent and water.

The aqueous phase of the compositions according to the invention that are in the form of an emulsion may comprise water, a floral water such as cornflower water, or a natural spring or mineral water, for example selected from eau de Vittel, the waters of the Vichy basin, eau d'Uriage, eau de la Roche Posay, eau de la Bourboule, eau d'Enghien-les-Bains, eau de Saint Gervais-les-Bains, eau de Néeris-les-Bains, eau d'Allevard-les-Bains, eau de Digne, eau de Maizières, eau de Neyrac-les-Bains, eau de Lons-le-Saunier, Eaux Bonnos, eau de Rochefort, eau de Saint Christau, eau des Fumades, eau de Tercis-les-Bains, eau d'Avène and eau d'Aix les Bains.

Said aqueous phase may be present at a content of from 10% to 90% by weight relative to the total weight of the composition, preferably from 20% to 80% by weight.

Non-limiting examples of gelling agents include those of the polyacrylamide family such as the sodium acryloyidimethyltaurate copolymer/isohexadecane/polysorbat 80. mixture marketed under the trademark Simulgel™ 600 by SeppiC™, the polyacrylamide/C13-14 isoparaffin/laureth-7 mixture, for instance the product marketed under the trademark Sepigel 305™ by Seppiet™, the family of acrylic polymers coupled to hydrophobic chains, such as the PEG-150/decyl/SMDI copolymer marketed under the trademark Aculyn 44™ (polycondensate comprising at least, as components, a polyethylene glycol containing 150 or 180 mol of ethylene oxide, decyl alcohol and methylenebis (4-cyclo-hexyl isocyanate) (SMDI), at 35% by weight in a mixture of propylene glycol (39%) and water (26%)), and the family of modified starches such as the modified potato starch marketed under the trademark Structure Solanace™, or mixtures thereof.

The preferred gelling agents are from the polyacrylamide family, such as Simulgel 600™ or Sepigel 305™, or mixtures thereof.

The gelling agent as described above may be used at preferred concentrations ranging from 0.1% to 15%, and more preferably ranging from 0.5% to 5%. The gels may preferably be prepared by dispersing or dissolving metronidazole in an appropriate ratio in a gel of carbomer, poloxamer or cellulose-based type.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

A specific formulation in accordance with the invention in the form of a gel for topical application is illustrated here:

| Metronidazole | 0.75 | g |
|---|---|---|
| Azelaic acid | 2 | g |
| Carbopol 980 (GOODRICH) | 0.6 | g |
| Polyethylene glycol 400 | 3 | g |
| Sodium hydroxide | qs pH 5 | |
| Preservatives | qs | |
| Deminoralized water | qs 100 | g |

### EXAMPLE 2

A specific formulation in accordance with the invention in the form of a cream for topical application use is illustrated here:

| Metronidazole | 0.75 | g |
|---|---|---|
| Methyl glucose sesquistearate | 1 | g |
| Stearyl alcohol | 0.5 | g |
| Liquid petroleum jelly | 6 | g |
| Polyethylene glycol 400 | 2 | g |
| Polyoxyethylenated methyl glucose | 5 | g |
| sesquistearate comprising 20 mol of EO | | |
| Carbopol 981 (GOODRICH) | 0.4 | g |
| Glycerol | 7 | g |
| Azelaic acid | 6 | g |
| Cyclomethicone | 4 | g |
| Sodium hydroxide | qs pH 5 | |
| Preservatives | qs | |
| Demineralized water | qs 100 | g |

### EXAMPLE 3

A specific formulation in accordance with the invention in the form of a lotion for topical application is illustrated here:

| | | |
|---|---|---|
| Metronidazole | 1 | g |
| Azelaic acid | 2 | g |
| Propylene glycol | 30 | g |
| Alcohol | 40 | g |
| Water | qs 100 | g |

### EXAMPLE 4

A specific formulation in accordance with the invention in the form of a cream for topical application is illustrated here:

| | | |
|---|---|---|
| Metronidazole | 1.5 | g |
| Azelaic acid | 3 | g |
| Methylparaben | 0.1 | g |
| Propylparaben | 0.1 | g |
| Lanolin | 5 | g |
| Liquid petroleum jelly | 4 | g |
| Sesame oil | 4 | g |
| Cetyl alcohol | 5 | g |
| Glyceryl monostearate | 2 | g |
| Triethanolamine | 1 | g |
| Propylene glycol | 5 | g |
| Carbomer 940 | 0.1 | g |
| Water | qs 100 | g |

### EXAMPLE 5

A specific formulation in accordance with the invention in the form of an ointment for topical application is illustrated here:

| | | |
|---|---|---|
| Metronidazole | 2 | g |
| Azelaic acid | 5 | g |
| Glycerol monostearate | 3 | g |
| Propylene glycol | 12 | g |
| Petrolatum | 82.9 | g |
| Water | qs 100 | g |

### EXAMPLE 6

A specific formulation in accordance with the invention in the form of a gel for topical application is illustrated here:

| | | |
|---|---|---|
| Metronidazole | 0.75 | g |
| Azelaic acid | 3 | g |
| Hydroxypropylcellulose | 1 | g |
| PPG-12-Buteth-16 | 2 | g |
| Triethanolamine | 0.2 | g |
| Propylene glycol | 5 | g |
| Alcohol | 45 | g |
| Carbomer 940 | 0.2 | g |
| Water | qs 100 | g |

### EXAMPLE 7

A specific formulation in accordance with the Invention in the form of an oil-in-water emulsion for topical application is illustrated here:

| | | |
|---|---|---|
| Metronidazole | 1 | g |
| Azelaic acid | 3 | g |
| Glyceryl stearate | 2 | g |
| Polysorbate 60 | 1 | g |
| Stearic acid | 1.4 | g |
| Triethanolamine | 0.7 | g |
| Carbomer | 0.4 | g |
| Olive oil | 12 | g |
| Liquid fraction of shea butter | 12 | g |
| Octylododecanol | 6 | g |
| Isononyl isononanoate | 10 | g |
| Antioxidant | 0.05 | g |
| Fragrance | 0.5 | g |
| Preservatives | 0.3 | g |
| Water | qs 100 | g |

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable pharmaceutical composition useful for the treatment of rosacea, comprising synergistically effective amounts of each of (i) metronidazole or derivative or salt thereof and (ii) azelaic acid or derivative or salt thereof, said composition having a concentration of azelaic acid of less than 10% by weight of the total weight thereof, formulated into a topically applicable pharmaceutical acceptable medium therefor.

2. The pharmaceutical composition as defined by claim 1, comprising from 0.001% to 20% by weight of said metronidazole or derivative or salt thereof.

3. The pharmaceutical composition as defined by claim 1, comprising from 0. 1% to 2% by weight of said metronidazole or derivative or salt thereof.

4. The pharmaceutical composition as defined by claim 1, comprising about 0.75% by weight of said metronidazole or derivative or salt thereof.

5. The pharmaceutical composition as defined by claim 1, comprising about 1% by weight of said metronidazole or derivative or salt thereof.

6. The pharmaceutical composition as defined by claim 1, comprising about 1.5% by weight of said metronidazole or derivative or salt thereof.

7. The pharmaceutical composition as defined by claim 1, comprising about 2% by weight of said metronidazole or derivative or salt thereof.

8. The pharmaceutical composition as defined by claim 1, comprising from 0.5% to 8% by weight of said azelaic acid or derivative or salt thereof.

9. The pharmaceutical composition as defined by claim 1, comprising from 1% to 7% by weight of said azelaic acid or derivative or salt thereof.

10. The pharmaceutical composition as defined by claim 1, comprising about 2% by weight of said azelaic acid or derivative or salt thereof.

11. The pharmaceutical composition as defined by claim 1, comprising about 3% by weight of said azelaic acid or derivative or salt thereof.

12. The pharmaceutical composition as defined by claim 1, comprising about 6% by weight of said azelaic acid or derivative or salt thereof.

13. The pharmaceutical composition as defined by claim 1, comprising about 7% by weight of said azelaic acid or derivative or salt thereof.

14. The pharmaceutical composition as defined by claim 1, formulated as an ointment, a cream, a lotion or a gel.

15. The pharmaceutical composition as defined by claim 1, formulated as an emulsion.

16. The pharmaceutical composition as defined by claim 1, further comprising at least one other bioactive agent selected from the group consisting of antibiotics, anti-bacterial agents, anti-viral agents, anti-parasitic agents, anti-fungal agents, anaesthetics, analgesics, anti-allergic agents, retinoids, free-radical scavengers, anti-pruriginous agents, keratolytics, anti-seborrheic agents, anti-histamines, sulfides, immunosuppressant or anti-proliferative products, and mixtures thereof.

17. The pharmaceutical composition as defined by claim 1, further comprising at least one pharmaceutical/dermatological additive selected from the group consisting of sequestering agents, antioxidants, sunscreens, preservatives, DL-alpha-tocopherol, fillers, electrolytes, humectants, dyes, inorganic or organic bases or acids, fragrances, essential oils, cosmetic active agents, moisturizers, surfactants, vitamins, essential fatty acids, sphingolipids, self-tanning compounds, agents that sooth and protect the skin, allantoin, propenetrating agents, gelling agents, and mixtures thereof.

18. A regime or regimen for the treatment of rosacea, comprising topically applying onto the affected skin area of an individual in need of such treatment, a thus effective amount of a topically applicable pharmaceutical composition comprising synergistically effective amounts of each of (i) metronidazole or derivative or salt thereof and (ii) azelaic acid or derivative or salt thereof, said composition having a concentration of azelaic acid of less than 10% by weight of the total weight thereof, formulated into a topically applicable pharmaceutical acceptable medium therefor.

19. A regime or regimen for the treatment of the inflammatory component of rosacea, comprising topically applying onto the affected skin area of an individual in need of such treatment, a thus effective amount of a topically applicable pharmaceutical composition comprising synergistically effective amounts of each of (i) metronidazole or derivative or salt thereof and (ii) azelaic acid or derivative or salt thereof, said composition having a concentration of azelaic acid of less than 10% by weight of the total weight thereof, formulated into a topically applicable pharmaceutical acceptable medium therefor.

20. The regime or regimen as defined by claim 18, comprising the treatment of the first stage of rosacea.

21. The regime or regimen as defined by claim 18, comprising the treatment of the second stage of rosacea.

22. The regime or regimen as defined by claim 18, comprising the treatment of the third stage of rosacea.

23. The regime or regimen as defined by claim 18, comprising the treatment of the fourth stage of rosacea.
